(19) United States
(12) Patent Application Publication
(10) Pub. No.: US 2025/0123343 A1
(43) Pub. Date: Apr. 17, 2025

(54) DEVICE FOR HOMOGENIZING A RADIOFREQUENCY FIELD FOR MAGNETIC RESONANCE IMAGING

(71) Applicants: UNIVERSITE D'AIX-MARSEILLE, Marseille (FR); CENTRE NATIONAL DE LA RECHERCHE SCIENTIFIQUE, Paris (FR); ECOLE CENTRALE DE MARSEILLE, Marseille (FR); MULTIWAVE TECHNOLOGIES, Marseille (FR)

(72) Inventors: Tania Del Socorro VERGARA-GOMEZ, Marseille (FR); Stefan ENOCH, Marseille (FR); Roxane MAMBERTI, Biver (FR); Redha ABDEDDAIM, Marseille (FR); Marc DUBOIS, Marseille (FR); Frank KOBER, Marseille (FR)

(73) Assignees: UNIVERSITE D'AIX-MARSEILLE, Marseille (FR); CENTRE NATIONAL DE LA RECHERCHE SCIENTIFIQUE, Paris (FR); ECOLE CENTRALE DE MARSEILLE, Marseille (FR); MULTIWAVE TECHNOLOGIES, Marseille (FR)

(21) Appl. No.: 18/717,761

(22) PCT Filed: Dec. 20, 2022
(86) PCT No.: PCT/EP2022/087107

§ 371 Date: Jun. 7, 2024

(30) Foreign Application Priority Data
Dec. 22, 2021 (FR) 2114224

(51) Int. Cl. G01R 33/34; G01R 33/565

(52) U.S. Cl. CPC G01R 33/34076; G01R 33/5659

(57) ABSTRACT

The invention relates to a device (2, 4) for homogenizing a radiofrequency field, for magnetic resonance imaging, of a given wavelength emitted by a volumetric antenna, comprising at least one continuous metal track (8) with a total length of between 50% and 75% of the wavelength of the radiofrequency field, the metal track comprising two end segments extending in mutually parallel directions and a main portion extending between the two end segments and comprising several local deformations identical to one another and connected to one another in series by at least one at least partially rectilinear connection portion (14) in such a way as to provide the homogenizing device (2, 4) with an electric dipole property, the homogenizing device having a fundamental frequency higher than the frequency corresponding to the wavelength of the radiofrequency field.

The present application relates to a radiofrequency field emission system for magnetic resonance imaging techniques.

Magnetic resonance imaging (MRI) machines generate a static main magnetic field using intense magnets, and a radio frequency (RF) excitation field using one or more transmitting antennas. The RF excitation field penetrates an object to be imaged (for example a human body or part of it) and interacts with atomic nuclei (for example protons) present in the object to be imaged in order to excite them. For this interaction to be optimal, the RF excitation field must be resonant with the atomic nuclei; to achieve this, the RF excitation field is emitted at a particular frequency called the Larmor frequency of the atomic nucleus involved.

The Larmor frequency is an increasing function of the main magnetic field; thus, for a proton (that is a hydrogen nucleus), it is around 64 MHz in a main magnetic field of 1.5 T, while for a field of 7 T it is around 300 MHz. As the atomic nuclei return to their equilibrium state, they emit an RF signal which is measured by the MRI scanner, providing the data needed to reconstruct an image of the object, known as the MRI image.

MRI machines are divided into “low-field” and “high-field” machines, with main magnetic fields of between 1.5 and 3 T, and “ultra-high-field” machines, with main magnetic fields of up to 7 T and more. The use of very intense magnetic fields enables a significant increase in the signal-to-noise ratio (SNR) of the RF signal measurement used to provide MRI images, but this is accompanied by an increase in the Larmor frequency, and therefore a reduction in the wavelength required for the RF excitation field.

Low-field and high-field clinical devices are equipped with a so-called body antenna to transmit the RF excitation field to the atomic nuclei to be studied, fairly homogeneously throughout the body.

The use of a body antenna is no longer possible in the case of ultra-high-field MRI machines, which require RF excitation fields whose wavelength is too short (11 cm in the human body at 300 MHZ) to be transmitted homogeneously throughout the body by a single antenna.

The strategy may then consist of using antennas dedicated to certain parts of the body (referred to as “volumetric antennas” in this description), such as the head, for which volumetric antennas of the “birdcage” type are used.

In this case, the volumetric antennas used ensure the transmission of the RF excitation field required for measurement in areas of reduced dimensions, but despite the reduction in the area to be imaged, the inhomogeneity problems explained above may remain. Such problems can also occur with high-field MRI applied to the trunk (thorax, abdomen or pelvis).

In order to improve the homogeneity of the RF excitation field (and therefore the quality of the image obtained thanks to the excitation induced by the RF field), it is known to use dielectric pads inserted between the volumetric antenna and the part of the body to be imaged, in order to distribute the RF excitation field homogeneously in said part of the human body.

Such dielectric pads can be made from a solvent, such as water, and particles of material with a higher dielectric constant to make them thinner and more comfortable to use. However, due to water evaporation and particle sedimentation, these dielectric pads have a relatively short service life. Their repeated handling during use (placement between the human body part and the volumetric antenna) accelerates particle sedimentation and therefore reduces the service life of the dielectric pads.

EP 3 550 321 describes a dielectric pad with a different composition. This is a dielectric pad comprising a polar solvent, a dispensing agent and a dielectric compound. The aim of this composition is to reduce the problem of particle sedimentation and improve particle distribution in the dielectric pad. However, these cushions are very bulky, making them difficult to use in the potentially small space between the imaging equipment and the part of the body to be imaged. Such dielectric pads run the risk of puncturing and spilling their liquid onto patients, thus creating a health hazard.

Finally, we know of the use of metasurfaces with negative magnetic permeability to concentrate the RF field like a magnetic lens. The metasurface can include a metal track that forms a magnetic dipole excited by the magnetic component of the electromagnetic field emitted by the microstrip antenna. However, these metasurfaces are best suited to surface antennas and are not optimal for use in a volumetric antenna, particularly of the birdcage type.

The present invention describes a system comprising one or more devices for homogenizing the RF excitation field for an MRI unit, enabling the above-mentioned difficulties to be overcome.

The object of the invention is a device for homogenizing a radiofrequency field, for magnetic resonance imaging, of a given wavelength emitted by a volumetric antenna, the homogenizing device comprising at least one continuous metal track with a total length of between 50% and 75% of the wavelength of the radiofrequency field, the metal track forming a pattern having a width of between 4% and 10% of the wavelength of the radiofrequency field and a height of between 10% and 25% of the length of the radiofrequency field, the metal track comprising two end segments extending in mutually parallel directions and a main portion extending between the two end segments and comprising several local deformations identical to one another and connected to one another in series by at least one at least partially rectilinear connection portion in such a way as to provide the homogenizing device with an electric dipole property, the homogenizing device having a fundamental frequency higher than the frequency corresponding to the wavelength of the radiofrequency field.

The use of a homogenizing device as described above makes it possible to redistribute the radiofrequency field emitted by a volumetric antenna of an MRI unit and thus improve the homogeneity of the distribution of the radiofrequency field in the part of the body to be imaged. Greater homogeneity of the radiofrequency field results in MRI images with better contrast.

The homogeneity device also does not interfere with the reception of the RF signal generated by atomic nuclei in the body part to be imaged by the reception channels of a surface antenna array of an MRI unit. Nor is the radiofrequency field increased to levels that are harmful to the body.

The use of several local deformations as described above, connected in series by at least one at least partially rectilinear portion, makes it possible to achieve homogenization in one or more zones of the part of the body to be imaged that is relatively far from the homogenizing device itself. In other words, the homogenizing device can be placed in one area and still achieve optimum results in a remote area. As a result, the homogenizing device can be attached to a volumetric antenna and still achieve the desired result. This eliminates the need to handle the homogenizing device each time an MRI scan is performed.

The use of several local deformations as described above, connected in series according to the invention to form a continuous structure, makes it possible to combine their electric dipole effect in order to improve the electric dipole behavior of the metal track, and thus improve the homogenization of the radiofrequency field by the homogenizing device.

Finally, the shape of the homogenizing device described above makes it possible to achieve an optimum compromise between the homogenizing device's own bandwidth and the homogenizing device's radiation efficiency. In other words, by arranging the metal track as described above, the widest possible bandwidth for the homogenizing device (to ensure that the operating frequencies overlap with the bandwidth of the volumetric antenna) is achieved while maintaining high radiation efficiency, where some devices choose to favor one or the other. For example, prior art devices can achieve a high quality factor but a narrow bandwidth (the quality factor being equal to the frequency at which the gain is at its highest, divided by the bandwidth), resulting in a homogenizing device that is difficult to couple to the volumetric antenna and highly sensitive to the local environment. Conversely, if the quality factor is too low, the result will be a homogenizing device with poor resonance and low correction efficiency.

In the present description, we will speak of the frequency of the radiofrequency field corresponding to the wavelength of the radiofrequency field and being related to the latter by the following equation:

$$f = c / λ \qquad [ Math 1 ]$$

Where f is the frequency of the radiofrequency field, λ is the wavelength of the radiofrequency field and c is the speed of light in a vacuum.

In this description, the frequency of the radiofrequency field is the Larmor frequency used in an MRI scanner.

According to the present description, the surface of the pattern is the surface of an area wherein the metal track is inscribed, for example a rectangular area or a square area.

According to other optional features of the homogenizing device, taken either alone or in combination:
The local deformations are placed at regular intervals;
The end segments are aligned;
The pattern is formed by several second-order Hilbert curves connected in series, several second-order Koch curves connected in series, or several second-order Minkowski curves connected in series. Among other things, these shapes enable the structure to resonate like an electric dipole at the Larmor frequency. The use of such curves makes it possible to maintain these resonance properties while folding the metal track of a given overall length;
The second-order Hilbert, second-order Koch, or second-order Minkowski curves are distributed in an alternating right/left pattern along several aligned, at least partially rectilinear connection portions;
At least one at least partially straight connection portion comprises a cross-shaped pattern. The presence of cross-shaped patterns enhances the performance of the homogenizing device;
The pattern is between 4 and 10 centimeters wide and between 10 and 25 centimeters high. These are the optimum size intervals for a static magnetic field strength of 7T;
The metal track is arranged on a dielectric substrate. This maintains the mechanical structure of the metal track;
The dielectric substrate is between 0.1 and 1 millimeter thick. This results in a thin, flexible structure that is easy to install, especially for antennas with complex geometries; and
The substrate has a dielectric loss factor, evaluated at the frequency corresponding to the wavelength of the radio-frequency field, of less than 0.05. This optimizes the efficiency of the homogenizing device by limiting the absorption of the radiofrequency field by the substrate. In the present description, the dielectric loss factor (or loss angle) is a dimensionless quantity with the meaning known to the skilled person in the microwave field. For a dielectric material, it is approximately equal to the ratio between the imaginary and real parts of the material's complex dielectric constant. The dielectric loss factor depends in particular on the frequency of the radiofrequency field considered.

Another object of the invention is an assembly formed by a volumetric antenna configured to emit a radiofrequency field and a homogenizing device according to the invention.

Advantageously, the volumetric antenna is a birdcage-type antenna configured to be placed around an area of the body to be imaged.

### BRIEF DESCRIPTION OF THE FIGURES

The invention will be better understood on reading the following description given solely by way of example and with reference to the appended drawings in which:

FIG. 1 shows two examples of a homogenizing device according to the invention;

FIG. 2 shows several examples of a metal track according to the invention;

FIG. 3 shows simulation results showing the distribution of the radiofrequency excitation field with an example of a homogenizing device according to the invention; and

FIG. 4 shows experimental results showing the distribution of the radiofrequency excitation field with an example of a homogenizing device implementing a bilateral configuration.

### DETAILED DESCRIPTION

It should be noted that, in the present description, some elements are not shown to scale, for reasons of visibility. In addition, and for reasons of visibility, only some of the elements with the same numerical reference are referenced.

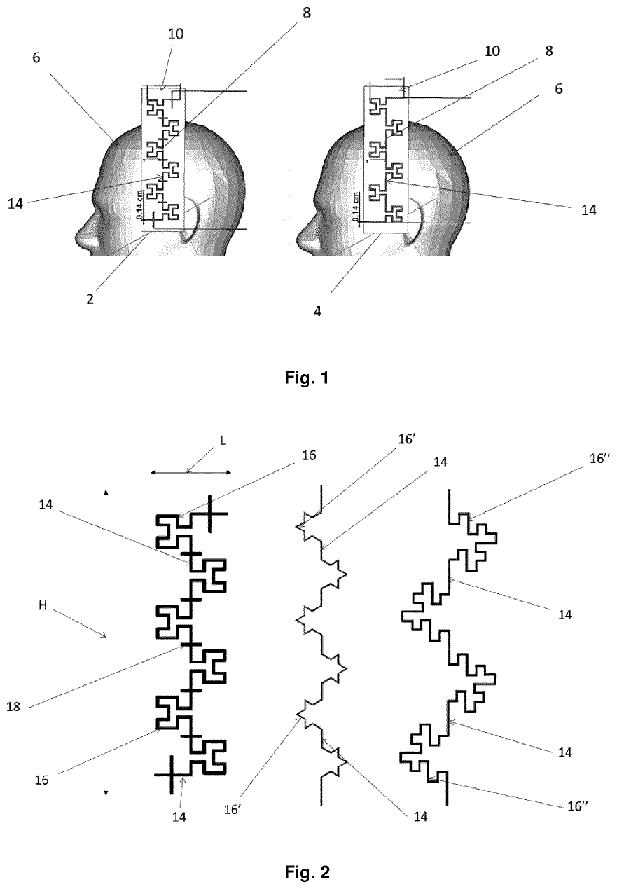

FIGS. 1 and 2 show examples of devices 2 and 4 according to the present invention. These homogenizing devices are designed for use in a radiofrequency field (hereinafter referred to as “RF field”) emission system for magnetic resonance imaging (MRI) equipment. A head 6 is shown schematically to show the positioning and order of magnitude of the dimensions of homogenizing devices 2 and 4 in relation to body parts.

The emission system comprises one or more RF field emission volumetric antennas and one or more RF field homogenizing devices. Such systems are configured to fit around a part of a body to be imaged (for example the head 6) and to emit, in this part of the body, a homogeneous RF field at a given frequency in order to excite atomic nuclei there.

The part of the body to be imaged may be placed on a support around which the volumetric antenna is positioned. Placement can be carried out by sliding the volumetric antenna along a placement slide.

The frequency of the RF field used to excite the atomic nuclei of the body part to be imaged, called the Larmor frequency, depends on the type of atomic nuclei to be excited and the main magnetic field of the MRI unit. The Larmor frequency can be, for example, around 300 MHz in the case of hydrogen atom nuclei in a main magnetic field of around 7 T. The wavelength of the RF field used can therefore be, for example, around 1 meter.

FIG. 1 shows two examples of a device for homogenizing a radiofrequency field according to the present invention, also known as RF field “homogenization pads”. In these examples, the devices comprise a metal track 8 arranged on a dielectric substrate 10. As will be described in more detail later, the metal track 8 comprises two end segments extending in directions parallel to each other (the end segments being aligned with each other in examples shown in FIG. 2) and a main portion extending between the two end segments and comprising several local deformations (that is at least two) identical to each other, placed at regular intervals in the examples described and connected to each other in series by at least one at least partially rectilinear connection portion 14 in such a way as to provide the homogenizing device with an electric dipole property.

Each local deformation is contained in a single two-dimensional plane containing the entire structure forming it, and forms an open curve that does not intersect with itself or with any other local deformation. The curves of the local deformations can form angles or be curved portions.

The presence of two end segments surrounding several local deformations, made at regular intervals and respecting a symmetry around the center of the pattern, makes it possible to obtain a maximum amplitude at the center of the pattern and therefore to have a centered magnetic field (in fact, the current at the end segments is low, but we observe an increase in the resonance frequency at the local deformations).

The metal track 8 is continuous, with a total length (by “total length” of the metal track 8 we mean the length of the fully unfolded track) preferably between 50% and 75% of the wavelength of the RF field used by the MRI unit. In practice, we aim to ensure that the metal track occupies a sufficiently large surface area to cover the part of the body to be imaged.

The metal track 8 forms a pattern with a width L of between 4% and 10% of the RF field wavelength and a height H of between 10% and 25% of the RF field length. FIG. 2 shows the height H and width L of a metal track on an example of a metal track according to the invention. These are respectively the distance between the two points furthest apart in a longitudinal direction of the metal track, and the distance between the two points furthest apart in a transverse direction of the metal track. For example, and for an electromagnetic wave length in vacuum equal to 1 meter, the width L of the pattern is between 4 centimeters and 10 centimeters and its height H is between 10 centimeters and 25 centimeters. These intervals are optimal for volumetric antennas used for brain MRI at 7T.

In particular, this dimension enables the surface to cover the part of the body to be imaged, such as a brain or pelvic area. In particular, when the device is applied to brain MRI, said dimensions ensure that the device is effective in the lateral or temporal lobes, which are areas wherein the RF field is generally not very present when a birdcage antenna is used without a homogenizing device.

In some cases, it may be necessary to image an area larger than the surface covered by a single metal track 8. The number of metal tracks 8 present on a dielectric substrate 10 can therefore vary. Each of the metal tracks 8 has dimensions within the above ranges.

Preferably, the metal track 8 forms (that is the pattern is formed by) several second-order Hilbert curves 16, according to their mathematical definition, connected in series as described above, preferably six second-order Hilbert curves. This shape enables the structure to resonate like an electric dipole at the Larmor frequency. The use of Hilbert curves makes it possible to maintain these resonance properties while folding the metal track of a given overall length. In practice, the use of Hilbert curve geometry means that a metal track of a given overall length can be folded to reduce the two-dimensional space it occupies on a surface, while retaining the same overall length (corresponding to the unwound length). In particular, this enables a metal track according to the invention to interact with bulk antennas emitting RF fields of a wavelength greater than the dimensions of the surface occupied by the metal track.

The second-order Hilbert curves 16 can be distributed alternately right/left along a number of aligned, at least partially rectilinear connection portions 14, and cross-shaped patterns 18 can be positioned at the at least partially rectilinear connection portions 14. The left/right alternation is shown in FIG. 1 on both homogenizing devices 2 and 4, with homogenizing device 2 comprising a cross pattern 18 on each connection portion 14 between two second-order Hilbert curves 16, the cross patterns 18 enhancing the performance of homogenizing device 2. The existence of symmetry helps to give the homogenizing device 2 or 4 the properties of an electric dipole that can interact with a volumetric antenna.

As an alternative to several second-order Hilbert curves, local deformations can be formed by second-order Koch curves 16′, or second-order Minkowski curves 16″, with the same effects as described above. The number of curves, their arrangement, or the presence of cross patterns can be identical to what has been described for second-order Hilbert curves 16.

The three examples of curves shown above shows preferential, but not limiting, cases of the possibilities of creating a pattern according to the invention.

The metal track 2 or 4 can be arranged, for example printed, on a dielectric substrate 10. This maintains the mechanical structure of the metal track. This dielectric substrate 10 is large enough to contain the metal track 2 or 4. Its thickness can be between 0.1 and 1 millimeter, for example 0.5 millimeters. This results in a thin, flexible structure that is easy to install, especially for antennas with complex geometries.

The dielectric substrate 10 may comprise a dielectric loss factor, evaluated at the frequency corresponding to the wavelength of the radio-frequency field, of less than 0.05. This optimizes the efficiency of the homogenizing device by limiting the absorption of the radiofrequency field by the substrate. In the present description, the dielectric loss factor (or loss angle) is a dimensionless quantity with the meaning known to the skilled person in the microwave field. For a dielectric material, it is approximately equal to the ratio between the imaginary and real parts of the material's complex dielectric constant. The dielectric loss factor depends in particular on the fr′quency of the radiofrequency field considered.

According to the invention, the homogenizing device 2 or 4 has a fundamental frequency higher than the frequency corresponding to the wavelength of the RF field, for example higher than a Larmor frequency used by an MRI unit (for example 300 MHz for a 7 T ultra-high field MRI unit or 125 MHz for a 3 T high field MRI unit). This prevents the presence of the human body from making the fundamental frequency lower than the RF field frequency when the device is in use. This allows the device to homogenize the RF field without resonating with it.

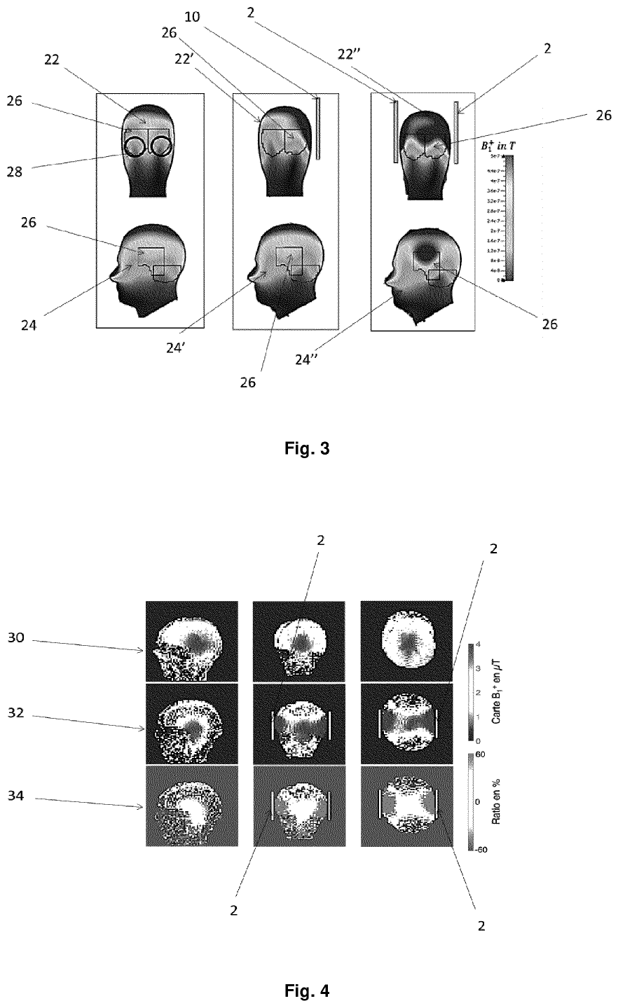

FIG. 3 shows simulation results for the distribution of the RF excitation field with an example of the device according to the present description.

In particular, the simulations model the distribution of a 300 MHz RF field (corresponding to that used in 7 T ultra-high-field MRI equipment) in the case of brain imaging.

The simulations use the parameters of a quadrature birdcage antenna comprising one (center column) or two (right column) homogenizing devices 2 or 4 (homogenizing device 2 is referenced in FIG. 3, but it could be reference 4 as well).

The head model used is a SAM (Specific Anthropomorphic Mannequin) phantom model traditionally used in this type of simulation. The SAM model is a dummy with standardized properties close to those of the human body (dielectric permittivity: 42, electrical conductivity: 0.99 S/m and density 1000 kg/m3).

Simulations are carried out using CST Microwave Studio® simulation software to assess the RF magnetic field distribution and the specific absorption rate (SAR). SAR (expressed as W/kg) quantifies the amount of electromagnetic power absorbed by human body tissues, which is then dissipated in the form of heat. This quantity is typically used by the skilled person to assess safety criteria for the use of radiation equipment on a patient.

In particular, FIG. 3 shows simulations of the RF field distribution in a brain in three cases:
without homogenizing device (left column),
with a single homogenizing device, in this example a homogenizing device 2 or 4, placed on a single side of the brain in the right temporal area (central column), and
with two homogenizing devices 2 or 4 (right-hand column).

The metal track 8 has dimensions within the above-mentioned ranges.

For each of the three configurations, the top median coronal plane (22, 22′, 22″) and the bottom median sagittal plane (24, 24′, 24″) are shown. Areas outlined in black indicate areas of interest 26 corresponding to different brain regions.

The areas, for example represented by arrow 28, indicate examples of shadow areas wherein the RF field is very weak due to a lack of RF field homogeneity. According to one or more embodiments, the device of the invention aims to eliminate these shadow areas 28 by homogenizing the distribution of the RF field throughout the part of the body to be imaged.

FIG. 3 shows that the addition of a homogenizing device according to the present invention eliminates shadow areas 28 in the brain by reintroducing signal through homogenization of the RF excitation field (this is clearly visible by comparing the median coronal planes 22 and 22″ but also the median sagittal planes 24 and 24″).

What's more, RF field homogeneity in the cerebellum area is not overly affected by the presence of homogenizing devices 2 or 4. This aspect is remarkable in that it differs from known pads in the prior art based on dielectric materials.

The results of statistical averages of RF field values in different brain areas for different device configurations are shown in Table 1 below.

TABLE 1

| | | Metasurface on | Metasurfaces |
|---|---|---|---|
| | Reference | just one side | on both sides |
| Left temporal area | 0.207 ± 0.071 | 0.185 ± 0.072 | 0.302 ± 0.134 |
| Right temporal area | 0.204 ± 0.076 | 0.258 ± 0.116 | 0.314 ± 0.1489 |

They show a clear improvement in the mean value of the RF field amplitude in the temporal areas (+45% for the unilateral configuration and +56% for the bilateral configuration).

The CST Microwave Studio® simulation software can also be used to calculate the power absorbed in the phantom model's brain. The quantity of interest is the specific absorption rate (SAR), whose value can be averaged over an entire volume of a part of the body (global value) or the maximum value in volumes equivalent to 10 g of tissue (local value).

Table 2 below shows the SAR results simulated for different configurations of the device according to different embodiments of the invention. The results in Table 2 show an increase of around 45% in local SAR in the case of the bilateral configuration.

This effect can be counterbalanced by a slight increase in the distance between the device and the head. It's a compromise between signal increase in shadow areas and local SAR.

Table 2 below shows that the local SAR for brain imaging remains low, even when the antenna and imaged areas are in close proximity.

TABLE 2

| | | Metasurface on | Metasurfaces on |
|---|---|---|---|
| | Reference | just one side | both sides |
| Overall SAR W · kg⁻¹ | 0.126 | 0.116 | 0.114 |
| Local SAR (10 g) | 0.571 | 0.520 | 0.833 |
| W · kg⁻¹ | | | |

Experimental tests showing the effect of the device on the distribution of a 300 MHz RF field in the context of brain MRI have also been carried out to verify the homogenization function of the device according to the present invention.

The measurements shown in FIG. 4 are the results of in vivo testing. The homogenizing devices 2 or 4 are positioned as in the tests shown in FIG. 3.

In these experimental tests, the antenna used is a quadrature birdcage antenna with a transmit/receive channel aimed at imaging the brain with a 7T ultra-high-field MRI unit. The metal track 8 is printed, its dimensions corresponding to those mentioned above.

FIG. 4 shows the experimental results for the RF excitation field distribution with an example of a device implementing a unilateral configuration, according to the present invention.

In particular, FIG. 4 shows the results of measuring the flip angle (a quantity proportional to the RF field amplitude) in the brain phantom model described above for different measurement configurations:
without a homogenizing device (top line 30), and
with two homogenizing devices (central line 32).

The third line 34 in FIG. 4 shows a comparison between the two above-mentioned lines in order to visualize the relative signal gain between the two configurations.

For each measurement configuration, results are presented in a sagittal section (left column), a coronal section (middle column) and an axial section (right column).

The results show a very positive effect of the device on RF field amplitude, with an increase of around 50% to 60% in each temporal lobe).

### LIST OF REFERENCES

2, 4: homogenizing devices
6: head
8: metal track
10: substrate
14: rectilinear connection portions
16: second-order Hilbert curves
16′: second-order Koch curves
18′: second-order Minkowski curves
18: cross patterns
22, 22′, 22″: median coronal plane
24, 24′, 24″: median sagittal plane
26: areas of interest
28: shadow areas
30: measurement without homogenizing devices
32: measurement with homogenizing devices
34: comparison of measurements
L: pattern width
H: pattern height

1. A device for homogenizing a radiofrequency field, for magnetic resonance imaging, of a given wavelength emitted by a volumetric antenna, the device comprising at least one continuous metal track with a total length of between 50% and 75% of the wavelength of the radiofrequency field, the metal track forming a pattern having a width area of between 4% and 10% of the wavelength of the radiofrequency field and a height of between 10% and 25% of the wavelength of the radiofrequency field, the metal track comprising two end segments extending in mutually parallel directions and a main portion extending between the two end segments and comprising several local deformations identical to one another and connected to one another in series by at least one at least partially rectilinear connection portion in such a way as to provide the homogenizing device with an electric dipole property, the homogenizing device having a fundamental frequency higher than the frequency corresponding to the wavelength of the radiofrequency field.

2. The device according to claim 1, wherein the pattern is formed by a plurality of second-order Hilbert curves connected to one another in series, several second-order Koch curves connected to one another in series, or several second-order Minkowski curves connected to one another in series.

3. The device according to claim 2, wherein the second-order Hilbert, second-order Koch or second-order Minkowski curves are distributed alternately right/left along several aligned, at least partially rectilinear connection portions.

4. The device according to claim 1, wherein at least one at least partially rectilinear connection portion comprises a cross pattern.

5. The device according to claim 1, wherein the pattern has a width of between 4 and 10 centimeters and a height of between 10 and 25 centimeters.

6. The device according to claim 1, wherein the metal track is arranged on a dielectric substrate.

7. The device according to claim 6, wherein the dielectric substrate has a thickness of between 0.1 millimeters and 1 millimeter.

8. The device according to claim 6, wherein the substrate comprises a dielectric loss factor, evaluated at the frequency corresponding to the wavelength of the radio frequency field, of less than 0.05.

9. An assembly formed by a volumetric antenna configured to emit a radiofrequency field and a device according to claim 1.

10. The assembly according to claim 9, wherein the volumetric antenna is a birdcage antenna configured to be placed around an area of the body to be imaged.
